(19) United States
(12) Patent Application Publication
(10) Pub. No.: US 2009/0227964 A1
(43) Pub. Date: Sep. 10, 2009

(54) Implantable Fluid Transfer Access Port

(76) Inventors: Paul DiCarlo, Middleboro, MA (US); Stephanie Dubay, Westford, MA (US); Jeff Gray, Lexington, MA (US)

Correspondence Address: FAY KAPLUN & MARCIN, LLP, 150 BROADWAY, SUITE 702, NEW YORK, NY, 10038, US

(21) Appl. No.: 12/367,927

(22) Filed: Feb. 9, 2009

Related U.S. Application Data
(60) Provisional application No. 61/027,217, filed on Feb. 8, 2008.

(51) Int. Cl. A61M 5/42; G08B 21/00

(52) U.S. Cl. 604/288.01; 340/665

(57) ABSTRACT

An implantable port comprises a port body forming a fluid reservoir and a septum sealing a proximal end of the reservoir in combination with a clicker member biased toward a first configuration, the clicker member being deformable via mechanical force applied thereto and configured so that, when subject to a force of at least a predetermined magnitude, the clicker member rapidly deforms away from the first configuration to a second configuration to provide one of an aural and tactile feedback to the user.

### PRIORITY CLAIM

The present application claims priority to U.S. Provisional Patent Application Ser. No. 61/027,217 filed on Feb. 8, 2008 entitled “Implantable Fluid Transfer Access Port” to Paul DiCarlo, Stephanie Dubay and Jeff Gray. The entire disclosure of the above-identified application is expressly incorporated herein by reference.

### BACKGROUND

Repeated penetrations of internal body structures by needles for fluid transfer can have significant consequences in the long term including degradation of the organ or vessel which is repeatedly accessed. For this reason, procedures requiring such repeated access are often facilitated by implanting a port into which needles are inserted. The port is fluidly coupled to the structure to be accessed, providing a path for fluid transfer which reduces trauma to the body structure (e.g., vessels such as veins, arteries, etc.). Prior to the insertion of a needle, the port must be located, for example, by palpation of the skin in the vicinity of the port and then the location of a needle penetrable septum of the port must be accurately located. When one or more ports have been implanted in a patient, an X-ray or a CT-scan may be necessary to identify a particular port suited to the procedure currently being performed.

### SUMMARY OF THE INVENTION

The present invention relates to a implantable port, comprising a port body forming a fluid reservoir and a septum sealing a proximal end of the reservoir in combination with a clicker member biased toward a first configuration, the clicker member being deformable via mechanical force applied thereto and configured so that, when subject to a force of at least a predetermined magnitude, the clicker member rapidly deforms away from the first configuration to a second configuration to provide one of an aural and tactile feedback to the user.

### BRIEF DESCRIPTION OF THE DRAWINGS

The present invention is illustrated in the accompanying drawings in which:

FIG. 1 shows a cross-sectional side view of a vascular access port according to an embodiment of the invention in a first state;

FIG. 2 shows a partially cross-sectional side view of the port of FIG. 1 in a second state;

FIG. 3 shows a proximal view of the port of FIG. 1;

FIG. 4 shows a proximal view of a port according to a further embodiment of the invention; and

FIG. 5 shows a cross-sectional view of a port according to a still further embodiment of the invention.

### DETAILED DESCRIPTION

The present invention, which may be further understood with reference to the following description and the appended drawings, relates to implanted fluid transfer port and a means for identifying a type of such a port after implantation within a body. It is noted that although exemplary embodiments of the present invention are described below with respect to percutaneous procedures, the description is not meant to limit the application of the invention, which may be employed in a plurality of medicinal and non-medicinal fields.

Presently available vascular access ports are identified by, for example, unique shapes identifiable through palpation. However, the intervening skin and tissue can make accurate differentiation between the shapes of the various ports and proper identification of these ports difficult.

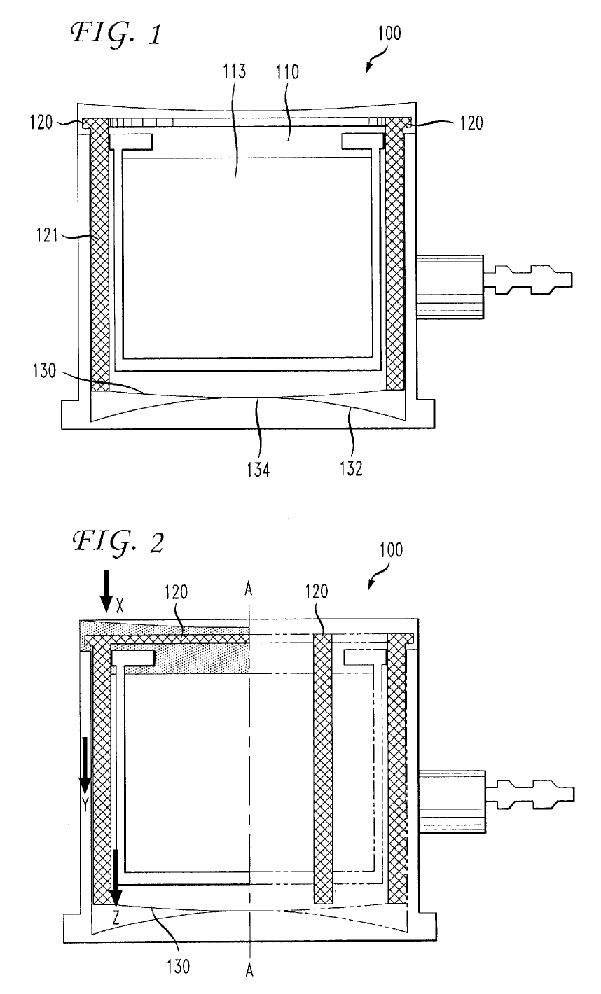

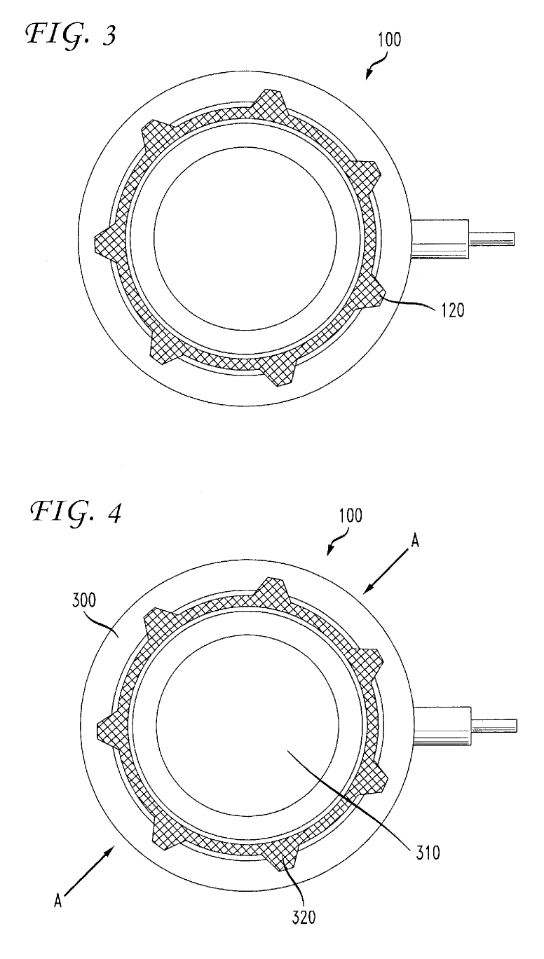

A device according to the present invention employs a port comprising a clicking mechanism which serves to identify a type of and location of the port. As shown in FIG. 1, an implanted vascular access port 100 according to an exemplary embodiment of the present invention comprises a septum 110 to seal a fluid chamber 113 formed within the port 100. The septum 110 may be comprised of a penetrable, self-sealing material such as silicone, as known in the art, to seal the fluid chamber 113 and prevent the passage of fluids between the port 100 and surrounding tissues. When implanted in the body, the septum 110 of the port 100 is preferably positioned to face the skin to facilitate insertion of a needle therethrough. The port 100 may further include a clicker actuator ring 120 extending around at least a portion of a periphery of the port 100. As further shown in the top-view of FIG. 3, the clicker actuator ring 120 may, for example, be formed as a ring encircling a proximal end of the port 100 radially outside the septum 110 with a clicker actuator member 121 extending through the port 100 toward a distal end thereof. A distal end of the clicker actuator member 121 contacts an outer periphery of a clicker member 130 which extends across the port 100 distal of a distal end of the reservoir 113. In another embodiment of the present invention, the clicker actuator member 121 and the clicker member 130 may be integrally formed or formed of separate elements bonded to one another or simply in contact with one another. The clicker member 130 is preferably formed as a thin sheet of material (e.g., a disk) biased toward a first position (e.g., concave proximally) in which it contacts a distal surface 132 of the port 100 only at a fulcrum point or fulcrmn path 134 with a space between the periphery of the clicker member 130 and the distal surface 132. Thus, moving the clicker actuator ring 120 distally relative to the port 100 pushes the clicker actuator member 121 against the periphery of the clicker member 130, thus bending the clicker member 130 about the fulcrum point or path 134 to produce an audible click.

For example, the clicker member 130 of the port 100 may be formed as a substantially parabolical disk with a thickness of the disk decreasing toward a central portion thereof. The clicker member 130 may be formed of a flexible material having a memory capability allowing the clicker member 130 to flex with the application of a force thereupon and return to its original position upon the removal of the force. Examples of suitable materials having memory capability include ELGILOY™, 304 spring stainless steel, a polymer or polymer alloy and other suitable materials as known in the art. Furthermore, the clicker member 130 may be formed in any suitable shape fitting within the confines of the port 100, such as a triangle, square, etc. so long as mechanical contact with the clicker actuator member 121 is maintained.

In accordance with an exemplary method of the present invention, a user wishing to make an injection, palpates a region of the skin under which one or more ports 100 are implanted and locates the clicker actuator ring 120. The person then holds the port 100 steady and applies a force to the clicker actuator ring 120 in the direction X shown in FIG. 2 to force the clicker actuator member 121 distally as indicated by the arrow Y. This distal movement generates a click via the resulting deformation of the clicker member 130 in the direction of the arrow Z from a concave shape (viewed from the proximal side) to a convex shape. The clicking capability or lack thereof gives the user information as to the identity of the port 100 being palpated. For example, the user may know that, of the two ports implanted in a patient, only the power injection suitable port 100 has clicking capability. Upon release of the palpation force, the clicker member 130 snaps back to its resting concave position which may also generate a clicking sound as well as a short reverberating pulse that may be felt by the user of the port 100.

In yet another embodiment of the present invention, the port 100 may comprise any plurality of units, such as an actuator comprising two or more pieces engaging one another. It is noted that although any plurality of units may be employed in this embodiment, a minimum of the three core units is required, including the septum 110, the clicker actuator ring 120 and the clicker member 130.

As shown in FIG. 4, a port 300 according to a further embodiment of the present invention comprises a shape memory ring 320 that may be embedded in a radially outer rim of a septum 310. When a compressive force is applied to the septum 310 (e.g., radially compressive in the direction of arrows A) via palpation as described above, the shape memory ring generates an audible or tactile feedback response that identifies the port 300 (e.g., the type of port and/or the anatomical structure to which it is coupled). As would be understood by those skilled in the art, the ring 320 will resist compression until a sufficient force is applied to overcome the bias of the shape memory material at which point, the portions of the ring 320 being compressed will give way and rapidly switch from convex to concave producing the tactile and/or audible feedback. After this compressive force has been removed, the shape memory properties of the ring 320 restore the ring 320 to its original shape. In another embodiment, the shape memory ring may be embedded in the external body portion of the port 100. The shape memory ring may be covered by a polymer sealant applied thereupon during the manufacturing process. Similarly, when a compressive force is applied to the shape memory ring, an audible or tactile response may be elicited, indicating a location of the port 100 in the body.

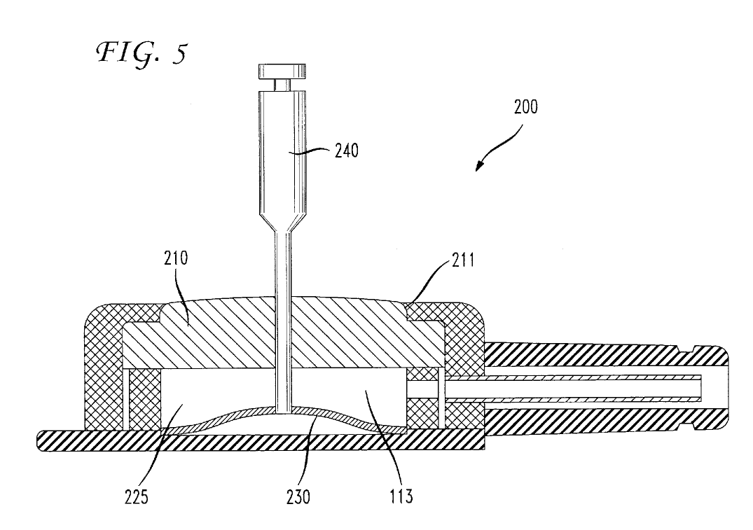

As shown in FIG. 5, a port 200 according to yet another embodiment of the invention provides audible feedback when manipulated to facilitate identification of the port 200. The port 200 comprises a septum 210 which covers an opening 211 located thereupon. Like the septum 110 of the embodiment of FIGS. 1-3, the septum 210 is formed of a material which reseals itself after penetration by a needle to maintain the port 200 sealed between injections. A needle or other device inserted through the septum 210 of port 200 is received into a reservoir 225, which is fluidly connected to, for example, a vein, duct, artery, vessel or organ in a body. Furthermore, a distal portion of the port 200 comprises a plate 230 composed, for example, as a plate shaped in a plane substantially transverse to a proximal-distal axis of the port 200 to correspond to a shape of the port 200 (e.g., circular) made up of, for example, titanium or any other suitable material. The plate 230 curves away from a distal surface of the port 200 to form a convex surface (as viewed proximally) with a portion of the plate 230 approaching a center thereof projecting proximally into the reservoir 225 while a perimeter portion of the plate 230 is attached to a distal end of the port 200 by any suitable means known in the art such as, for example, welding, adhesive, fasteners, interference fit, etc.

Accordingly, when a needle, such as needle 240 is inserted through the septum 210, a distal end of the needle 240 contacts the plate 230. The application of force driving the needle 240 further distally after this contact deflects the surface of the plate 230 with the resulting deformation of the plate 230 generating a clicking sound. The user may then use the presence or absence of this clicking capability to determine whether this is the desired port before operating a syringe or other device attached to the needle 240 to inject fluids to or withdraw fluids from the port 200. As would be understood by those skilled in the art, the plate 230 is preferably formed to automatically retract to the original convex position upon the removal of the force from the needle 240 so that, at the next injection, the same clicking sound may be generated.

As would be understood by those skilled in the art, the plate 230 may comprise any suitable shape (e.g., non-circular shapes or any other shapes corresponding to the shape of the port) and may be formed of any of a variety of suitable materials. For example, the plate 230 may be triangular, rectangular, etc. with the plate 230 placed over any portion of the distal end of the port 200 suited to achieve the desired deformation of the plate 230 upon palpation.

Those skilled in the art will understand that the described exemplary embodiments of the present invention may be altered without departing from the spirit or scope of the invention. Thus, it is to be understood that these embodiments have been described in an exemplary manner and are not intended to limit the scope of the invention which is intended to cover all modifications and variations of this invention that come within the scope of the appended claims and their equivalents. The specifications are, therefore, to be regarded in an illustrative rather than a restrictive sense.

What is claimed is:

1. An implantable port, comprising:
a port body forming a fluid reservoir:
a septum sealing a proximal end of the reservoir; and
a clicker member biased toward a first configuration, the clicker member being deformable via mechanical force applied thereto and configured so that, when subject to a force of at least a predetermined magnitude, the clicker member rapidly deforms away from the first configuration to a second configuration to provide one of an aural and tactile feedback to the user.

2. The port according to claim 1, further comprising an actuator coupled between the proximal end of the port and the clicker member, the clicker member being formed as a plate a portion of which abuts a fulcrum surface about which the clicker member is deformed so that, force applied to the actuator is applied to the clicker member to deform the clicker member about the fulcrum surface from the first configuration to the second configuration.

3. The port according to claim 2, wherein the fulcrum surface is formed as a part of a distal wall of the port.

4. The port according to claim 1, wherein the clicker member is formed as a ring at the proximal end of the port, radial compression of the ring toward a distal/proximal axis of the port causing the ring to deform from the first configuration to the second configuration.

5. The port according to claim 3, wherein the clicker member is formed of a shape memory material with a memorized shape of the clicker member being the first configuration so that, after a deforming force is released, the clicker returns to the first configuration.

6. The port according to claim 2, wherein a distal end of the actuator is in mechanical contact with a periphery of the clicker member.

7. The port according to claim 2, wherein the clicker member is formed as a substantially parabolical disk with a thickness that decreases toward a central point thereof

8. The port according to claim 1, wherein the clicker member forms at least a portion of a distal surface of the reservoir arched over a surface of the port body distal thereof so that mechanical force applied thereto via a needle inserted through the septum into the reservoir deforms the clicker member distally to the second configuration generating the one of an aural and tactile feedback.

9. The port according to claim 8, wherein the clicker member is formed of titanium.

10. An implantable port, comprising:
a port body forming a fluid reservoir;
a septum sealing a proximal end of the reservoir;
a clicker actuator extending around at least a portion of a periphery of the port; and
a clicker member abutting at least a portion of the clicker actuator, the clicker actuator transmitting to the clicker member mechanical force applied thereto wherein, when the mechanical force is at least a predetermined magnitude, the clicker member rapidly deforms away from the first configuration to a second configuration to provide one of an aural and tactile feedback to the user.

11. The port according to claim 10, wherein the clicker member extends distally of a distal surface of the reservoir.

12. The port according to claim 10, wherein, in the first configuration, a proximal surface of the clicker is concave and a distal surface thereof is convex.

13. The port according to claim 12, wherein a distal wall of the port includes a convex proximal surface abutting the convex distal surface of the clicker member at a fulcrum portion.

14. The port according to claim 13, the clicker actuator abuts a portion of the clicker member radially away from the fulcrum portion.
